(19) United States
(12) Patent Application Publication
(10) Pub. No.: US 2026/0062374 A1
(43) Pub. Date: Mar. 5, 2026

(54) PROCESS FOR THE PREPARATION OF A BENZENE DERIVATIVE

(71) Applicant: MELETIOS THERAPEUTICS, PARIS (FR)

(72) Inventors: Xavier MANIERE, Montrouge (FR); Stefano BERNA, Paris (FR)

(21) Appl. No.: 19/106,790

(22) PCT Filed: Aug. 26, 2023
(86) PCT No.: PCT/EP2023/073457

§ 371 Date: Feb. 26, 2025

(30) Foreign Application Priority Data
Aug. 26, 2022 (EP) 22315192.9

(51) Int. Cl. C07C 209/68; C07C 45/68

(52) U.S. Cl. CPC C07C 209/68; C07C 45/68; C07C 2601/14

(57) ABSTRACT

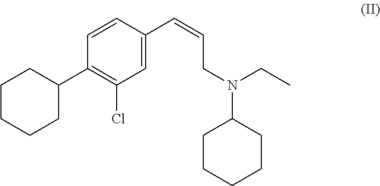 [SMILES: CCN(C/C=C\c1ccc(C2CCCCC2)c(Cl)c1)C1CCCCC1]

The present invention relates to a process for the preparation of N-[(Z)-3-(3-chloro-4-cyclohexylphenyl)prop-2-enyl]-N-ethylcyclohexanamine or a salt thereof of the following formula (II).

### Field of the invention

The present invention relates to a process for the preparation of N-[(Z)-3-(3-chloro-4-cyclohexylphenyl)prop-2-enyl]-N-ethylcyclohexanamine or a salt, ester, hydrate, derivative, prodrug or metabolite thereof.

### BACKGROUND OF THE INVENTION

N-[(Z)-3-(3-chloro-4-cyclohexylphenyl)prop-2-enyl]-N-ethylcyclohexanamine hydrochloride, also known as SR-31747, is a sigma ligand which has immunosuppressive activity blocking the proliferation of lymphocytes. Low concentrations of SR-31747 were shown to inhibit proliferation in yeast and in several human breast and prostate cancer cell lines both in vitro and in vivo, suggesting that it has cancer therapy potential.

The synthesis of SR-31747 has been described in EP376850 in four steps including:
(i) the formation of 3-chloro-4-cyclohexylacetophenone semicarbazone from 3-chloro-4-cyclohexylacetophenone of the following formula (a):

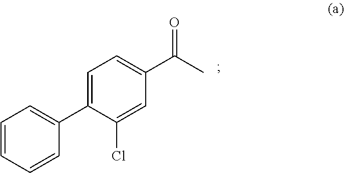 [SMILES: CC(=O)c1ccc(-c2ccccc2)c(Cl)c1]

(ii) the formation of 3-chloro-4-cyclohexyl-1-ethynylbenzene of the following formula (b) by reacting the semicarbazone with a selenium oxide

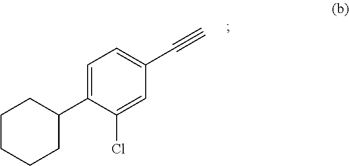

(iii) the cupper-catalysed Mannich reaction on terminal alkyne of 3-chloro-4-cyclohexyl-1-ethynylbenzene of formula (b) to introduce cyclohexylethylamine and form the N-[3-(3-chloro-4-cyclohexylphenyl)prop-2-ynyl]-N-ethylcyclohexanamine of the following formula (c):

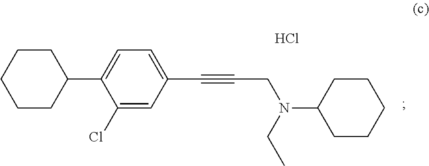 [SMILES: Cl ; CCN(CC#Cc1ccc(C2CCCCC2)c(Cl)c1)C1CCCCC1]

(iv) the hydrogenation reaction in presence of a metallic catalyst on support to form the N-[(Z)-3-(3-chloro-4-cyclohexylphenyl)prop-2-enyl]-N-ethylcyclohexanamine.

Synthesis of SR-31747 is usable in a laboratory but has numerous drawbacks for industrialization. Indeed, this approach suffers from the difficulty to supply the starting acetophenone with high and reproducible purity. In addition, selenium oxide is a very toxic reagent.

Thus, there is a need of a method for the preparation of the compound N-[(Z)-3-(3-chloro-4-cyclohexylphenyl)prop-2-enyl]-N-ethylcyclohexanamin or a salt thereof which is easy to implement, executable in an industrial scale, and safer for the human and the environment.

### SUMMARY OF THE INVENTION

The present invention arises from the unexpected finding, by the inventors, of a synthetic route to SR-31747 in a high purity and high yield in two steps from the commercially available 3,4-dichlorobenzaldehyde. This approach involved a regioselective Negishi cross-coupling with cyclohexylzinc chloride and a Z-selective Wittig olefination with an aminophosphonium. Furthermore, this synthetic route to SR-31747 is suitable for industrial-scale production.

Thus, the present invention relates to a process for the preparation of N-[(Z)-3-(3-chloro-4-cyclohexylphenyl)prop-2-enyl]-N-ethylcyclohexanamine or a pharmaceutically acceptable salt, ester, hydrate, derivative, prodrug or metabolite thereof of the following formula (II):

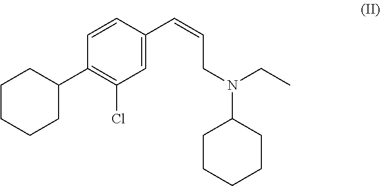 [SMILES: CCN(C/C=C\c1ccc(C2CCCCC2)c(Cl)c1)C1CCCCC1]

characterized in that it comprises a step (i) of reacting a compound of the following formula (1):

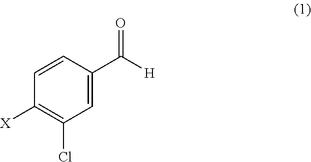

wherein X represents a chloride, a bromide, an iodine, a triflate or an acetyloxy group, with an organozinc compound of the following formula (2):

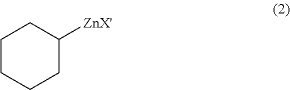

wherein X′ represents a chloride, a bromide, or an iodine group, to form the 3-chloro-4-cyclohexylbenzaldehyde of the following formula (I):

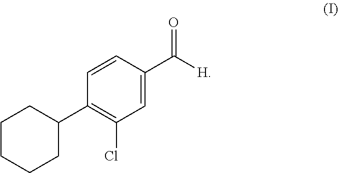

The present invention also relates to a process for the preparation a compound of the following formula (I):

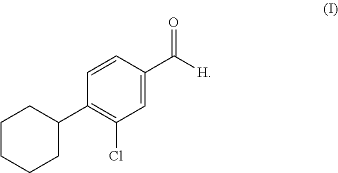

comprising a step of reacting a compound of the following formula (1):

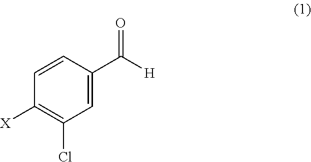

wherein X represents a chloride, a bromide, an iodine, a triflate or an acetyloxy group, with an organozinc compound of the following formula (2):

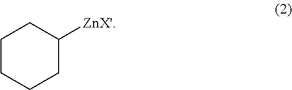

The present invention also relates to the use of a compound of the following formula (I):

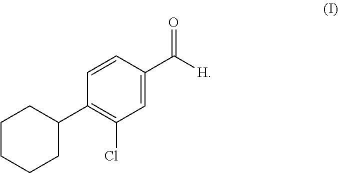

for the reparation of N-[(Z)-3-(3-chloro-4-cyclohexylphenyl)prop-2-enyl]-N-ethylcyclohexanamine of formula (II) or a pharmaceutically acceptable salt, ester, hydrate, derivative, prodrug or metabolite thereof.

The present invention also relates to a compound of formula (II) or a salt, ester, hydrate, derivative, prodrug or metabolite thereof, obtained by the process according to the invention.

The present invention also relates to a compound of formula (II) or a salt, ester, hydrate, derivative, prodrug or metabolite thereof, obtained by the process according to the invention for use as a drug.

The present invention also relates to a method for the prevention or treatment of a disease in an individual, comprising administering to the individual a prophylactically or therapeutically effective quantity of at least one compound of formula (II) or a salt, ester, hydrate, derivative, prodrug or metabolite thereof.

The present invention also relates to the use of a compound of formula (II) or a salt, ester, hydrate, derivative, prodrug or metabolite thereof for the manufacture of a medicament for the prevention or treatment of a disease in an individual.

The present invention also relates to a pharmaceutical composition comprising as active substance a compound of formula (II) or a salt, ester, hydrate, derivative, prodrug or metabolite thereof.

### DETAILED DESCRIPTION OF THE INVENTION

As intended herein, the word “comprising” is synonymous to “including” or “containing”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

As intended herein, the term “in situ” means in the reaction mixture. As intended herein, the term “about” defines a range of plus or minus 10% of the cited value.

The halogen according to the invention is preferably selected from the group consisting of Cl, Br, F and I. More preferably, the halogen atom according to the invention is a chlorine (Cl).

The halide according to the invention is preferably selected from the group consisting of a fluoride (F³¹ ), a chloride (Cl⁻), a bromide (Br⁻), and an iodide (I⁻).

As intended herein, the terms “alkyl” or “alkyl group” refer to a linear or branched saturated group containing 1 to 12 carbon atoms.

As intended herein, the terms “aryl” or “aryl group”, refer to a functional group or substituent derived from an aromatic ring containing 3 to 12 carbon atoms.

As intended herein, the sulfonate group is a group having the following formula −S(═O)₂−O⁻.

As intended herein the term “triflate” is synonymous to trifluoromethanesulfonate and is a functional group with the formula CF₃SO₃−.

As intended herein, the term “acetyloxy group” is synonymous to acetoxy group, abbreviated AcO or OAc, and is a chemical functional group of the structure CH₃−C(═O)−O⁻.

As intended herein, the term “organozinc compound” refers to a compound containing a carbon to zinc chemical bond.

As intended herein, the expression “organic solvent” refers to carbon-based substances capable of dissolving or dispersing one or more other substances.

Preferably, the process according to the invention is monitored by any method well known to the person skilled in the art which allows the identification and quantification of organic substances. By way of example of method which can be used to monitor the process according to the invention, it is possible to cite high-performance liquid chromatography such as UPLC, HPLC, UPLC-MS, UPLC-MS/MS, etc. Purities of the substance according to the present inventions have been determined by UPLC (Ultra Performance Liquid Chromatography). Yield of the reaction according to the present invention have been measured by UPLC.

### Process for the Preparation of N-[(Z)-3-(3-Chloro-4-Cyclohexylphenyl)Prop-2-Enyl]-N-Ethylcyclohexanamine

### Step (i)

Step (i) provides a compound of the following formula (I) which is a key intermediate in the preparation of N-[(Z)-3-(3-chloro-4-cyclohexylphenyl)prop-2-enyl]-N-ethylcyclohexanamine according to the invention:

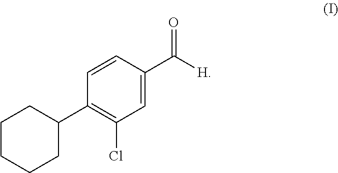

Preferably, the molar yield of step (i) is at least 60%, at least 65%, at least 70%, at least 75%, at least 80%, at least 85%, at least 90%, at least 95%.

Preferably, the molar yield of step (i) is comprised between 70% and 99%, 75% and 99%, 80% and 99%, 85% and 99%, 90% and 99%, 95 and 99%, or 98 and 99%.
Preferably, the compound of formula (I) is obtained with a purity of at least 50%, at least 55%, at least 60%, at least 65%, at least 70%, at least 75%, at least 80%, at least 85%, at least 90%, or at least 95%.

Preferably, compound of formula (I) is formed with a purity of from 50% to 95%, 55% to 90%, 55% to 85%, more preferably, from 60% to 95%, 65% to 90%, 70% to 90%, 75% to 90%, or 80% to 90%.

Preferably, in the compound of formula (1), X is Cl. The compound of formula (1) wherein X is Cl is known as 3,4-dichlorobenzaldehyde. This compound is well known to the person skilled in the art and is commercially available.

Compound 3,4-dichlorobenzaldehyde is of the following formula (1a):

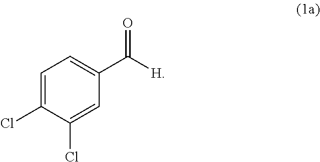

Preferably, in step (i) compound of formula (1) according to the invention reacts with the organozinc compound according to the invention in an amount of from 1.1 to 2 molar equivalents, for example from 1.1 to 1.8 molar equivalents, 1.1 to 1.7 molar equivalents, 1.1 to 1.6 molar equivalents, 1.1 to 1.5 molar equivalents, 1.1 to 1.4 molar equivalents, 1.1 to 1.3 molar equivalents, 1.1 to 1.2 molar equivalents, more preferably from 1.2 to 1.5 molar equivalents.

Preferably, the organozinc compound according to the invention of the following formula (2)

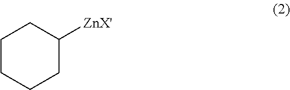

is formed in situ by reacting a cyclohexyl magnesium salt of the following formula (4):

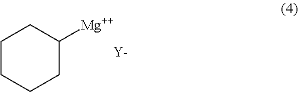

wherein Y³¹ represents a halide,
with a zinc salt of the following formula (5):

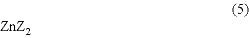

wherein Z represents a halide.

Preferably, Y⁻represents a fluoride (F⁻), a chloride (Cl⁻), a bromide (Br⁻), or a iodide (I⁻). More preferably, Y-represents a chloride (Cl⁻).

Preferably, Z represents a fluoride (F⁻), a chloride (Cl⁻), a bromide (Br⁻), or a iodide (I⁻). More preferably, Z represents a chloride (Cl⁻).

Preferably, the cyclohexylmagnesium salt according to the invention is a cyclohexylmagnesium chloride. Cyclohexylmagnesium chloride is well known to the person skilled in the art and is commercially available.

Preferably, the zinc halide according to the invention is a zinc chloride.

Preferably, the organozinc compound according to the invention is a cyclohexylzinc chloride.

Preferably, step (i) according to the invention is performed in the presence of a catalyst.

Preferably, the catalyst according to the invention is a transition metal catalyst. The transition metal catalyst can be chosen among any transition metal catalyst well known by the person skilled in the art.

Preferably, the transition metal catalyst is a palladium catalyst.

Preferably, the palladium catalyst according to the invention is selected from the group consisting of a palladium-phosphine ligand complex, a palladium carbene catalyst, and a palladium dimer catalyst.

More preferably, the palladium catalyst is selected from the group consisting of Buchwald catalyst, in particular the third generation of Buchwald catalyst, Pd(OAC)2-XPhos, Pd21I2(PtBu2)2, Pd-PEPSI-IPent, Pd-PEPPSI-iPr, and a compound of the following formula (6)

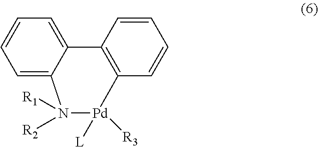

wherein:
R₁ and R₂ independently represents, H, or a C₁-C₁₂ alkyl group;

R3 represents a C1-C12 alkyl group, a C3-C12 aryl group, a halogen or a C1-C12 alkyl sulfonate group;

L represents a ligand of the type XPhos, SPhos, RuPhos, or CPhos.
Preferably, in the compound of formula (6),
   R₁ represents a H,
   R₂ represents a H,
   R₃ represents a C₁-C₁₂ alkyl sulfonate group, more preferably, a methanesulfonate (C1 alkyl sulfonate group, CH₃SO₃−); and
   L represents a ligand of the type XPhos, SPhos, RuPhos, or CPhos.
Preferably, the catalyst according to the invention is present in an amount of 0.5 to 10 mol %, preferably 1 to 9 mol %, 1 to 8 mol %, 1 to 7 mol %, 1 to 6 mol %, 1 to 5 mol %, 1 to 4 mol %, 1 to 3 mol %, or 1 to 2 mol %.

Preferably, step (i) according to the invention is performed at a temperature comprised between 10 and 60° C., for example 10 to 55° C., 15 to 50° C., 15 to 45° C., 15 to 40° C., 15 to 35° C., 15 to 30° C., 15 to 25° C., 15 to 20° C., or 20 to 55° C., 25 to 55° C., 30 to 55° C., 35 to 55° C., 40 to 55° C., or 45 to 55° C. More preferably, step (i) is performed at a temperature comprised between 15 and 25° C.

Preferably, step (i) according to the invention is performed in a solvent, more preferably an organic solvent.

The organic solvent for performing step (i) can be chosen among any suitable organic solvent well known to the person skilled in the art in the field of organic chemistry. Preferably, the organic solvent according to the invention is selected from the group consisting of tetrahydrofurane (THF), dimethoxymethane, 1,2-dimethoxyethane, toluene, acetonitrile, dichlomethane, acetone and mixture thereof.

### Step (ii)

Preferably, the process according to the invention, further comprises a step (ii) of reacting the compound of formula (I) with a phosphonium salt of the following formula (3):

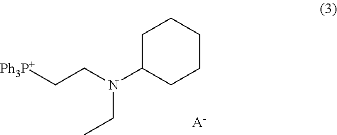

wherein A⁻represents a halide.

Preferably, A⁻represents a fluoride (F⁻), a chloride (Cl⁻), a bromide (Br⁻), or an iodide (I⁻). More preferably, A-represents a bromide (Br⁻).

Preferably, step (ii) provides the compound of formula (II) in a free base form, also known as SR-31747 free base.

In step (ii) the phosphonium salt is preferably present in an amount of about 1.1 to 3 molar equivalents, for example from 1.1 to 2.5, 1.1 to 2, 1.1 to 1.9, 1.1 to 1.8, 1.1 to 1.7, 1.1 to 1.6, 1.1 to 1.5, 1.1 to 1.4, 1.1 to 1.3, or 1.1 to 1.2 molar equivalents.

Preferably, the conversion in step (ii) is of at least 70% to up to 100%, for example from 75% to 100%, 80% to 100%, 85 to 100%, 86% to 100%, 87 to 100%, 88% to 100%, 89% to 100%, 90% to 100%, 91% to 100%, 92% to 100%, 93% to 100%, 94% to 100%, 95% to 100%, 96% to 100%, 97% to 100%, 98% to 100%, or 99% to 100%.

Preferably, step (ii) according to the invention provides the compound N-[3-(3-chloro-4-cyclohexylphenyl)prop-2-enyl]-N-ethylcyclohexanamine in a Z/E ratio of from 60/40 to 99/1, from example of about 65/35, 70/30, 75/25, 80/20, 85/15, 86/14, 87/13, 88/12, 89/11, 90/10, 91/9, 92/8, 93/7, 94/6, or 95/5.

Preferably, step (ii) according to the invention provides the compound of formula (II) in a purity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 97%, at least 98%, or at least 99%.

Preferably, step (ii) according to the invention is performed in the presence of a base. The base according to the invention can be chosen among any suitable base well known by the person skilled in the art. Preferably, the base according to the invention is selected from the group consisting of potassium bis (trimethylsilyl) amide (KHMDS), n-butyllithium, tert-butyllithium, potassium ter-butoxide, Lithium diisopropylamide (LDA), Lithium diethylamide (LDEA), lithium tetramethylpiperidide (LTPM), and potassium carbonate.

Preferably, the base according to the invention is present in an amount from 1.1 to 3 molar equivalents, for example 1.1 to 2.5, 1.1 to 2.4, 1.1 to 2.3, 1.1 to 2.2, 1.1 to 2.1, 1.1 to 2, 1.1 to 1.9, 1.1 to 1.8, 1.1 to 1.7, 1.1 to 1.6, 1.1 to 1.5, 1.1 to 1.4, 1.1 to 1.3, 1.1 to 1.2 molar equivalents.

Preferably, step (ii) is performed in a solvent, more preferably an organic solvent.

The organic solvent for performing step (ii) can be chosen among any suitable organic solvent well known to the person skilled in the art. Preferably, the organic solvent according to the invention is selected from the group consisting of tetrahydrofurane (THF), dimethoxymethane, 1,2-dimethoxyethane, toluene, acetonitrile, dichlomethane, acetone and mixture thereof.

Preferably, step (ii) according to the invention is performed at a temperature comprised between o and 60° C., for example 10 to 55° C., 15 to 50° C., 15 to 45° C., 15 to 40° C., 15 to 35° C., 15 to 30° C., 15 to 25° C., 15 to 20° C., or 20 to 55° C., 25 to 55° C., 30 to 55° C., 35 to 55° C., 40 to 55° C., or 45 to 55° C.

More preferably, step (ii) is performed at a temperature comprised between 15 and 25° C.

In an embodiment, step (ii) is performed at reflux temperature.

The phosphonium slat of formula (3) according to the invention can be prepared by any method well known to the person skilled in the art. By way of example, the phosphonium salt of formula (3) according to the invention can be prepared from a vinylphosphonium salt, preferably a vinylphosphonium bromide, in a solution of cyclohexylethylamine.

Preferably, the process for the preparation of the phosphonium slat of formula (3) according to the invention is performed in an organic solvent. The organic solvent suitable for the preparation of the phosphonium salt of formula (3) according to the invention can be chosen among any common organic solvent well known by the person skilled in the art in the field of organic chemistry. By way of example, the organic solvent suitable for the preparation of the phosphonium salt of formula (3) according to the invention is preferably selected from the group consisting of acetonitrile, diethyl ether, diisopropyl ether, methyl tert-butyl ether, dichloromethane, ethyl acetate, chloroform, THF, methyl isobutyl ketone, pentane, heptane, cyclohexane, and mixture thereof.

Preferably, the product obtained by reacting a vinylphosphonium salt with a cyclohexyethylamine is further dispersed in an organic solvent, such as for example diethyl ether, diisopropyl ether, to provide after filtration the phosphonium salt of formula (3).

Preferably, the phosphonium salt of formula (3) is obtained in a yield of at least 75%, at least 80%, at least 85%, or at least 90%.

Preferably, the phosphonium salt of formula (3) is obtained in a purity of at least 75%, at least 80%, at least 85%, at least 90%, at least 95%, at least 96%, at least 97%, at least 97%, or at least 99%.

Preferably, the cyclohexyethylamine is present in an amount of from about 1.1 to 5 molar equivalents, for example from 1.1 to 4.5, 1.1 to 4, 1.1 to 3.5, 1.1 to 3, 1.1 to 2.5, 1.1 to 2, 1.1 to 1.5, 1.1 to 1.4, 1.1 to 1.3, or 1.1 to 1.2 molar equivalents.

Preferably, the reaction of a vinylphosphonium salt with a cyclohexyethylamine according to the invention is performed at a temperature comprised between 15 and 85° C., such as for example from 15° C. to 20° C., 15° C. to 25° C., 15° C. to 30° C., 15° C. to 35° C., 15° C. to 40° C., 15° C. to 45° C., 15° C. to 50° C., 15° C. to 55° C., 15° C. to 60° C., 15° C. to 65° C., 15° C. to 70° C., 15° C. to 75° C., 15° C. to 80° C., or 15° C. to 85° C. More preferably, the reaction of a vinylphosphonium salt with a cyclohexyethylamine according to the invention is performed at a temperature comprised between 15° C. and 25° C.

In an embodiment, the reaction of a vinylphosphonium salt with a cyclohexyethylamine according to the invention is performed at a temperature of about 20° C., about 25° C., about 30° C., about 35° C., about 40° C., about 45° C., about 50° C., about 55°, about 60° C., about 65° C., about 70° C., about 75° C., about 80° C. or about 85° C.

In an embodiment, the reaction of a vinylphosphonium salt with a cyclohexyethylamine according to the invention is performed at reflux temperature.

Step (iii)

The process according to the invention further comprises a step (iii) of hydrolysis.

The step (iii) of hydrolysis can be performed by any method well known to the person skilled in the art. Preferably, the step (iii) of hydrolysis is performed in an aqueous layer, more preferably, in an aqueous acid layer.

The aqueous acid layer according to the invention can be chosen among any aqueous acid layer known to the person skilled in the art. By way of example, aqueous acid layer according to the invention can comprise hydroiodic acid, hydrobromic acid, hydrochloric acid, acetic acid.

Preferably, the aqueous acid layer according to the invention is an aqueous HCI solution.

### Step (iv)

The process according to the invention further comprises a step (iv) of crystallisation.

The step (iv) of crystallisation can be performed by any conventional crystallisation method well known to the person skilled in the art.

Preferably, the crystallization is performed in a solvent selected from the group consisting of acetonitrile, THF, acetone, toluene, ethyl acetate, isopropyl, and diisopropyl ether. More preferably, the crystallization is performed in a solvent selected from the group consisting of acetonitrile, THF, acetone, and toluene.

Preferably, step (iv) provides the compound of formula (II) according to the invention in a purity of at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 97%, at least 98%, or at least 99%.

Preferably, step (iv) provides the compound of formula (II) according to the invention in a yield of at least 40%, preferably at least 50%, at least 55%, at least 60%, at least 65%, at least 70%, at least 75%, or at least 80%.

### Compound of Formula (II)

N-[(Z)-3-(3-chloro-4-cyclohexylphenyl)prop-2-enyl]-N-ethylcyclohexanamine, also known as SR-31747 free base, is well known to one of skilled in the art and is represented by the following formula (II):

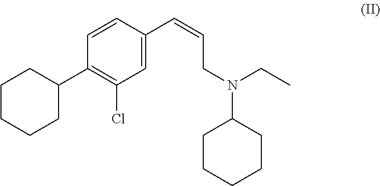 [SMILES: CCN(C/C=C\c1ccc(C2CCCCC2)c(Cl)c1)C1CCCCC1]

Pharmaceutically acceptable salt, ester, hydrate, derivative, prodrug or metabolite of the compound of formula (II) will be apparent to one of skilled in the art.

By way of example of pharmaceutically acceptable salt according to the invention, it is possible to cite salt of mineral or organic acids such as picric acid, oxalic acid, mandelic acid, camphosulphonic acid, as well as hydrochloride salt, hydrobromide salt, succinate salt, sulphate salt, hydrogen sulfate salt, dihydrogen phosphate salt, methanesulphonate salt, methyl sulphate salt, acetate salt, benzoate salt, citrate salt, glutamate salt, maleate salt, fumarate salt, p-toluenesulfonate salt, and 2-naphthalenesulfonate salt.

Preferably, the pharmaceutically acceptable salt of the compound of formula (II) is the hydrochloride salt, also known as SR-31747 or N-Cyclohexyl-N-ethyl-3-(3-chloro-4-cyclohexylphenyl)propen-2-ylamine hydrochloride.

The term “prodrug” as used herein refers to drug precursors which following administration to the individual, release the drug via chemical and/or physiological process e.g. by hydrolysis and/or enzymatic conversion. Prodrug according to the invention can easily be prepared by the person skilled in the art from the compound of formula (II) according to the invention using methods commonly used in the art. By way of example of common prodrugs, it is possible to cite acid derivatives, amides derivatives, acylated base, phosphorus-containing derivatives, etc.

The compound of formula (II), or ester, hydrate, derivative, prodrug or metabolite thereof obtained by the process according to the invention is preferably for use as a drug.

Preferably, the compound of formula (II), or ester, hydrate, derivative, prodrug or metabolite thereof obtained by the process according to the invention is for use in the prevention or treatment of a disease selected from the group consisting of a cancer, in particular prostate cancer and breast cancer, an infection by a virus, an autoimmune disease such as rheumatoid arthritis, and an inflammatory disease.

More preferably, the compound of formula (II), or salt, ester, hydrate, derivative, prodrug or metabolite thereof obtained by the process according to the invention is used as an antiviral agent.

In an embodiment, the compound of formula (II), or salt, ester, hydrate, derivative, prodrug or metabolite thereof according to the invention is used in combination or is combined with at least one additional compound intended for preventing or treating a disease selected from the group consisting of a cancer, in particular prostate cancer and breast cancer, an infection by a virus, an autoimmune disease such as rheumatoid arthritis, and an inflammatory disease.

The pharmaceutical composition comprising as active substance a compound of formula (II) or a salt, ester, hydrate, derivative, prodrug or metabolite thereof obtained by the process according to the invention is preferably for use in the prevention or treatment of a disease selected from the group consisting of a cancer, in particular prostate cancer and breast cancer, an infection by a virus, an autoimmune disease such as rheumatoid arthritis, and an inflammatory disease.

The pharmaceutical composition comprising as active substance a compound of formula (II) or a salt, ester, hydrate, derivative, prodrug or metabolite thereof obtained by the process according to the invention further comprise at least one additional compound intended for preventing or treating a disease selected from the group consisting of a cancer, in particular prostate cancer and breast cancer, an infection by a virus, an autoimmune disease such as rheumatoid arthritis, and an inflammatory disease.

The present invention is further illustrated with reference to the following examples. These examples are provided for illustrating the present invention and should not be construed as limiting the scope and spirit of the present invention.

### EXAMPLE

The inventors have synthesized N-[(Z)-3-(3-chloro-4-cyclohexylphenyl)prop-2-enyl]-N-ethylcyclohexanamine hydrochloride according to the following protocol.

### 1. Formation of Compound of Formula (I) by a Reaction of Negishi Coupling (step (i)

Cyclohexylzinc chloride is prepared in situ from a commercial 1,3 M solution of cyclohexylmagnesium chloride in 50/50 toluene/THF, added at room temperature to a THF solution of zinc chloride.

3,4-dichlorobenzaldehyde is added onto the mixture of organozinc reagent, followed by the addition of 2 mol % of palladium catalyst Pd-PEPPSI-iPr.

The reactions is monitored by UPLC.

The compound of formula (I) below is obtained in 82% purity. The yield of the reaction is 98%:

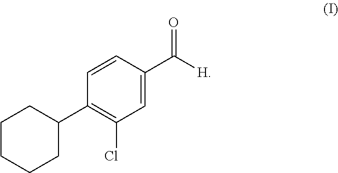

### 2. Wittig Reaction (Step (ii))

### 2.1 Preparation of the Beta-Aminophosphonium

Compound (3a) of the following formula:

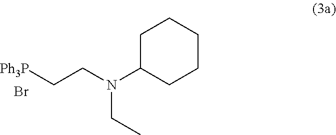

has been prepared from vinylphosphonium bromide in 3.5 molar equivalent of cyclohexylethylamine in dichloromethane (5 vol.). This resulted in the formation of a solid mass which was further dispersed in diisopropyl ether to provide after filtration the aminophsphonium of formula (3a) in 85% yield and 96% purity.
2.2. Formation of SR-31747 Free Base 3-chloro-4-cyclohexylbenzaldehdye from previous Negishi coupling is added to the mixture of aminophosphonium (3a) in 1.5 molar equivalent with tBuOk in 1.6 molar equivalent in THF.

This resulted in the formation of N-[(Z)-3-(3-chloro-4-cyclohexylphenyl)prop-2-enyl]-N-ethylcyclohexanamine.

### 3. Hydrolysis

Mixture comprising N-[(Z)-3-(3-chloro-4-cyclohexylphenyl)prop-2-enyl]-N-ethylcyclohexanamine is hydrolysed with an aqueous HCl solution (1 M).

This resulted in the N-[(Z)-3-(3-chloro-4-cyclohexylphenyl)prop-2-enyl]-N-ethylcyclohexanamine hydrochloride.

### 4. Crystallisation

N-[(Z)-3-(3-chloro-4-cyclohexylphenyl)prop-2-enyl]-N-ethylcyclohexanamine hydrochloride is recrystallized in ethyl acetate resulting in a product with 81% purity and 67% yield.

1. A process for the preparation of N-[(Z)-3-(3-chloro-4-cyclohexylphenyl)prop-2-enyl]-N-ethylcyclohexanamine or a salt thereof of the following formula (II): 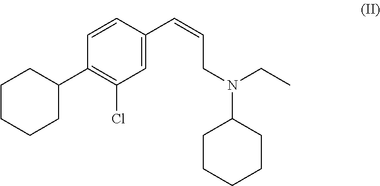 [SMILES: CCN(C/C=C\c1ccc(C2CCCCC2)c(Cl)c1)C1CCCCC1]
characterized in that it comprises a step (i) of reacting a compound of the following formula (1): 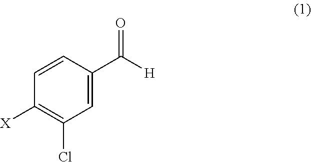
wherein X represents a chloride, a bromide, an iodine, a triflate or an acetyloxy group, with an organozinc compound of the following formula (2): 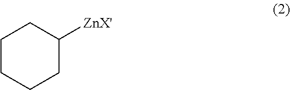
wherein X′ represents a chloride, a bromide, or an iodine group, to form the 3-chloro-4-cyclohexylbenzaldehyde of the following formula (I):

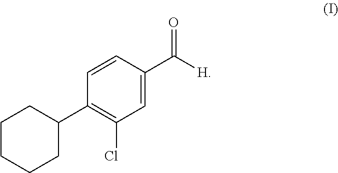

2. The process according to claim 1, further comprising a step (ii) of reacting the compound of formula (I) with a phosphonium salt of the following formula (3): 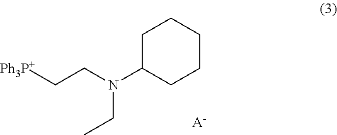
wherein A⁻represents a halide.

3. The process according to claim 1, further comprising a step (iii) of hydrolysis.

4. The process according to claim 1, further comprising a step (iv) of crystallisation.

5. The process according to claim 1, wherein step (i) is performed in the presence of a transition metal catalyst.

6. The process according to claim 5, wherein the transition metal catalyst is a palladium catalyst.

7. The process according to claim 6, wherein the palladium catalyst is selected from the group consisting of a palladium-phosphine ligand complex, a palladium carbene catalyst, and a palladium dimer catalyst.

8. The process according to claim 6, wherein the palladium catalyst is selected from the group consisting of Pd(OAC)2-XPhos, Pd212 (PtBu2)2, Pd-PEPSI-IPent, Pd-PEPPSI-iPr, and a compound of the following formula (6): 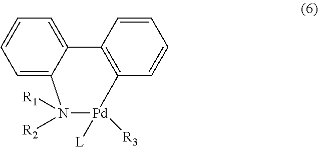
wherein
R₁ and R₂ independently represents, H, or a C₁-C₁₂ alkyl group;
R₃ represents a C₁-C₁₂ alkyl group, a C₃-C₁₂ aryl group, a halogen or a C₁-C₁₂ alkyl sulfonate group;
L represents a ligand of the type XPhos, SPhos, RuPhos, or CPhos.

9. The process according to claim 1, wherein step (ii) is performed in the presence of a base.

10. The process according to claim 9, wherein the base is selected from the group consisting of potassium bis(trimethylsilyl)amide, n-butyllithium, potassium ter-butoxide, and potassium carbonate.

11. The process according to claim 1, wherein step (i) and step (ii) are performed in an organic solvent.

12. The process according to claim 4, wherein the crystallization is performed in a solvent selected from the group consisting of acetonitrile, THF, acetone, and toluene.

13. The process according to claim 1, wherein the compound of the following formula (2): 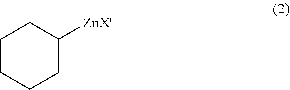
is formed in situ by reacting a cyclohexyl magnesium salt of the following formula (4): 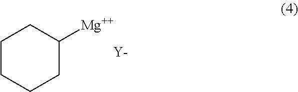
wherein Y⁻represents a halide,
with a zinc salt of the following formula (5):
ZnZ₂
(5)
wherein Z represents a halide.

14. The process according to claim 1, wherein
X is Cl,
X′ is Cl
A⁻is Br
Y⁻is Cl.

15. The process according to claim 1, wherein the compound of formula (II) is in the form of hydrochloride salt.
